(19) United States
(12) Patent Application Publication
(10) Pub. No.: US 2005/0102029 A1
(43) Pub. Date: May 12, 2005

(54) Total disc replacement system and related methods

(75) Inventor: Jason Blain, San Diego, CA (US)

Correspondence Address: ATTN: LEGAL DEPARTMENT;NU VASIVE, INC., 4545 TOWNE CENTRE COURT, SAN DIEGO, CA, 92121, US

(73) Assignee: Nu Vasive, Inc., San Diego, CA (US)

(21) Appl. No.: 10/975,268

(22) Filed: Oct. 28, 2004

Related U.S. Application Data
(60) Provisional application No. 60/515,247, filed on Oct. 28, 2003.

(51) Int. Cl. A61F 2/44

(52) U.S. Cl. 623/17.13

(57) ABSTRACT

A total disc replacement system for use in spine surgery involving a pair of endplates, a pair of bearing surfaces, and an intradiscal element composed of an elastomeric polymer.

### CROSS REFERENCES TO RELATED APPLICATIONS

The present application is a nonprovisional patent application claiming benefit under 35 U.S.C. § 119(e) from U.S. Provisional Application Ser. No. 60/515,247, filed on Oct. 28, 2003, the entire contents of which are hereby expressly incorporated by reference into this disclosure as if set forth fully herein.

### BACKGROUND OF THE INVENTION

I. Field of the Invention

The present invention relates generally to orthopedic surgery and, more particularly, to a total disc replacement system for use in spine surgery involving a pair of endplates, a pair of bearing surfaces, and an intradiscal element composed of an elastomeric polymer.

II. Discussion of the Prior Art

The human spine is a very delicate apparatus. The vertebral bone is separated and cushioned by intervertebral discs. When these discs rupture or otherwise deteriorate, back pain develops. Back pain can be minor or very serious, depending on the extent of injury to, or deterioration of, the disc.

Numerous U.S. patents disclose disc replacement inventions of different types intended to solve the problem. When simplified, most of these inventions are comprised of two endplates (or anchor plates) and an intradiscal element. In some variations, the two endplates are connected by a hinging mechanism to form one unit. The endplates function to attach the apparatus to the vertebral bone, while the intradiscal element functions to stabilize and cushion the adjacent vertebrae. With regard to injuries involving rupture of spinal ligaments, the intradiscal element can also serve the function of connecting adjacent vertebrae to each other.

Variations in prior inventions have occurred in two main ways: (1) means of attachment of the apparatus to the vertebrae and (2) the composition of the intradiscal element. To date, disc replacement apparatuses are attached to the vertebrae primarily through employment of a number of spikes on the surface of the mechanism. Another attachment method is use of a screw drilled directly into the bone. Screws will maintain their hold, but they tend to be larger in size and at least two are required to hold each plate in place.

To date, the types of intradiscal elements have varied widely between inventions. Some previous disc prostheses contained rigid assemblies primarily for the purpose of fusing the vertebrae together. Others use a hinging, or ball and socket mechanism to allow for limited motion. Some prior prostheses contained springs as an intradiscal element to act as a shock absorber. Still others seek to employ a polyolefin rubber material or a polyethylene plastic material as an intradiscal element. The idea behind this type is to allow for a greater array of motion while also maintaining some load bearing and shock absorbing functionality.

However, all of these previous attempts at invertebral disc prostheses are flawed in one way or another. Using spikes on the surface of the endplate to attach the prosthesis to the bone only prevents lateral movement of the device within the spinal column. Screws are better at securing the prosthesis to the bone, but are considerably bulkier and, as a result of boring deep holes and threads into the bone, may make it difficult to replace the prosthesis in the future, if such action is necessary.

Similarly, the various intradiscal elements previously employed can be easily improved upon. Rigid elements that serve no purpose other than to fuse adjacent vertebrae together are no longer desirable in most cases because they do not allow for the retention of motion. Semi-rigid elements such as a hinge or ball and socket mechanism allow for greater motion but are limited in the motion that they allow. The primary flaw is the absence of vertical elasticity: The mechanisms neither stretch nor provide any shock absorption if compressed. Rubber and plastic intradiscal elements seem to address this problem through their elasticity, but the problem with these devices is that the elements are firmly attached to the endplates. While they succeed at providing vertical elasticity as well as lateral bending motions, these elements must also resist torsional forces when the patient twists his/her body. In theory, a great torsional force could cause the intradiscal element to become detached from the endplate, and thus become detached from the vertebra.

The present invention is directed at overcoming, or at least improving upon, the disadvantages of the prior art.

### SUMMARY OF THE INVENTION

The present invention accomplishes this goal by providing a total disc replacement (TDR) mechanism that allows for vertical and lateral motion, provides a shock absorption function, and does not resist torsional forces.

According to one broad aspect of the present invention, the system comprises a total disc replacement mechanism for use in the human spine. The invention consists of a pair of endplates, a pair of bearing surfaces, and an intradiscal element composed of an elastomeric polymer.

The endplates may include any number of components capable of providing structural support and anchoring the mechanism to adjacent vertebrae. By way of example only, the endplates may be composed of a metal or ceramic material. The endplates may also be form-fitted to the size of the adjacent vertebrae. The anchor may include any number of components capable of anchoring the prosthesis to the vertebrae. By way of example only, the anchor may include a diametric structure protruding from the endplates at a generally perpendicular angle, optionally containing teeth to secure the prosthesis in place.

The bearing surfaces may include any number of components capable of providing a motile connection between the endplates and the intradiscal element. By way of example only, the bearing surfaces may include discs of approximately the same size as the intradiscal element, but smaller in diameter than the endplates. The bearing surfaces may be composed of a metal or ceramic material.

The intradiscal element may include any number of components capable of providing sufficient support yet also capable of flexing, bending, twisting, or elongating as the need may arise. By way of example only, the intradiscal element may include a naturally occurring or synthetic polymer that is nonreactive with the body, such as a hydrogel. Hydrogels may be cross-linked, for example by radiation or chemical treatment, to provide greater strength and thus improved functionality.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a perspective view of the total disc replacement system of the present invention, showing the showing two endplates with toothed anchors, two bearing surfaces, and the intradiscal element;

FIG. 2 is an exploded elevated perspective sectional view of the present invention, illustrating the knob-recess connection method of the endplates and bearing surfaces;

FIG. 3 is an elevated side view of the prosthesis of the present invention, showing the single perpendicular toothed anchor on each endplate.

FIG. 4 is the view of FIG. 3 rotated 90°, showing the diametrical orientation of the toothed anchor structure.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The total disc replacement system and related methods disclosed herein boasts a variety of inventive features and components that warrant patent protection, both individually and in combination.

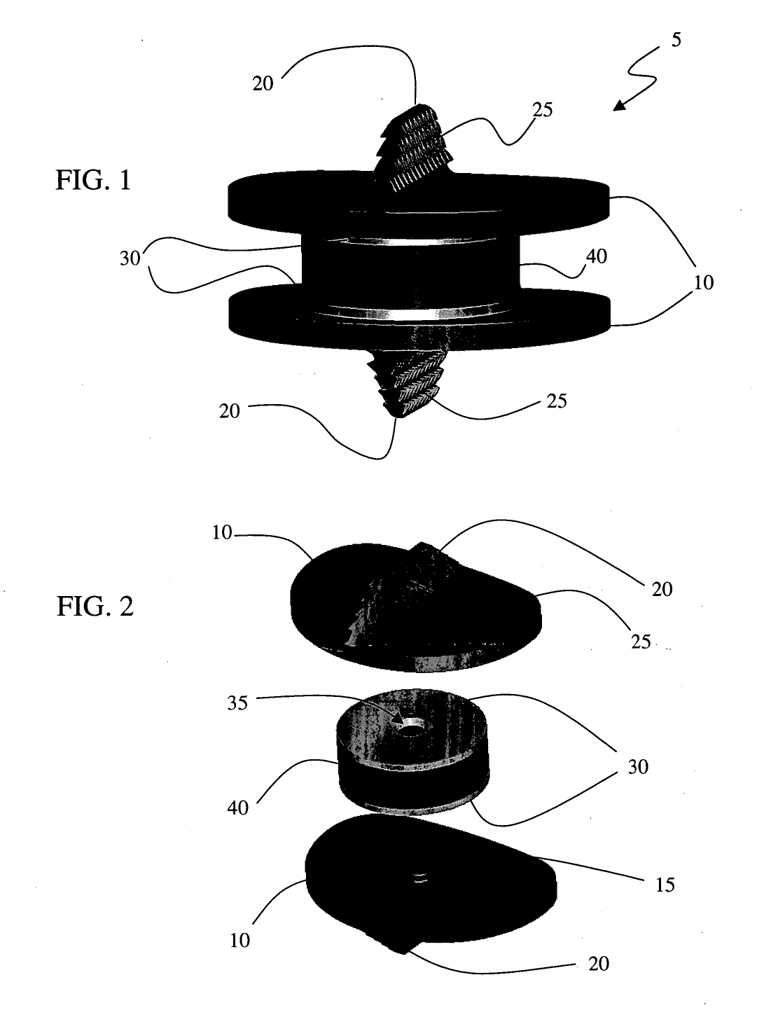

FIG. 1 illustrates a total disc replacement system 5 of the present invention. The total disc replacement system 5 includes two endplates 10, each endplate 10 containing one anchor structure 20, protruding in a generally perpendicular direction therefrom. The anchor 20 is comprised of a structure situated along the diameter of the endplate 10, extending in a generally perpendicular fashion and containing several rows of teeth 25. On the interior side of each endplate 10 is a bearing surface 30, which functions as an intermediate between the endplate 10 and the intradiscal element 40, allowing for greater mobility and a reduction in resistance to torsional forces.

FIG. 2 is an exploded perspective view of the present invention, illustrating the connection between the endplates 10 and bearing surfaces 30. The center of the interior face of each endplate 10 contains a knob 15 which fits into the center hole 35 of the bearing surface 30. This arrangement, once the device is fully assembled, will prevent the intradiscal element 40 from sliding laterally out of position.

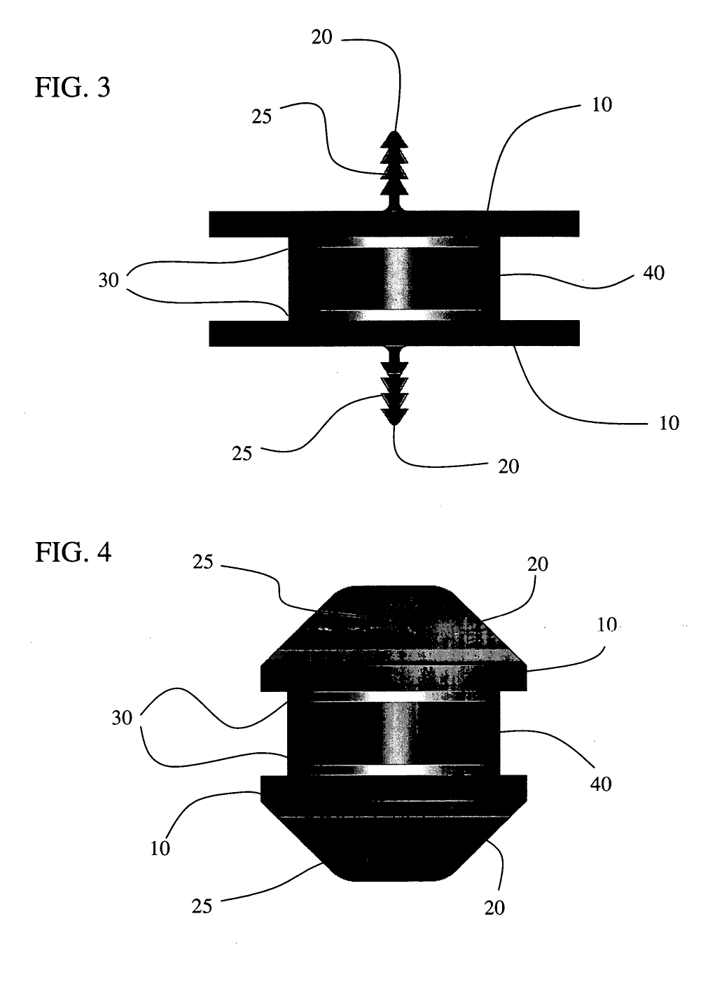

FIG. 3 demonstrates the location and orientation of the anchor mechanism 20 on the exterior side of each endplate 10, as well as the presence of teeth 25 on the anchor 20. The anchor 20 is situated along the diameter of the endplate 10 and extends in a perpendicular manner from the surface of the endplate 10. FIG. 3 shows four rows of teeth 25 on each anchor 20, but this configuration may be varied depending on the length of the anchor 20. The teeth 25 function to prevent displacement of the device once placed in the vertebrae.

FIG. 4 illustrates the elongated structure of the anchor 20, which extends perpendicularly from the surface of the endplate 10. The length of the base of the anchor 20 is equal to the diameter of the endplate 10. The anchor 20 is tapered as it extends from the endplate, such that the length of the side opposite the endplate 10 is somewhat less than the diameter of the endplate 10. The elongated structure and orientation of the anchor 20 functions to prevent lateral movement of the prosthesis once inserted into the vertebra. Such an orientation of the anchor 20 also prevents any twisting or rotating of the endplates that may have occurred in the absence of such a design.

1. A total disc replacement system, comprising:
a first anchor plate having a first and second generally flat surface;
a second anchor plate having a first and second generally flat surface; and
an internal assembly disposed between said first and second anchor plates, said internal assembly including a first bearing surface, a second bearing surface, and an elastomeric polymer.

2. The total disc replacement system of claim 1, wherein said first and second anchor plates each include an anchor element for placement into a first and second vertebral body, respectively.

3. The total disc replacement system of claim 2, wherein said anchor elements include a plurality of antimigration features.

4. The total disc replacement system of claim 3, wherein said plurality of antimigration features comprises a plurality of teeth.

5. The total disc replacement system of claim 2, wherein said anchor elements protrude in a generally perpendicular direction from said first surfaces of said first and second anchor plates.

6. The total disc replacement system of claim 1, wherein said first and second anchor plates each include a post extending generally perpendicularly from said second surfaces.

7. The total disc replacement system of claim 6, wherein said first and second bearing surfaces each include an aperture dimensioned to receive said post.

8. The total disc replacement system of claim 1, wherein said elastomeric polymer comprises at least one of a naturally occurring polymer, a synthetic polymer, and a mixture of naturally occurring and synthetic polymers.

9. The total disc replacement system of claim 1, wherein said elastomeric polymer comprises a hydrogel.

10. The total disc replacement system of claim 9, wherein said hydrogel is cross-linked.

11. The total disc replacement system of claim 1, wherein said first and second anchor plates are composed of at least one of ceramic and metal.

12. The total disc replacement system of claim 1, wherein said first and second bearing surfaces are composed of at least one of ceramic and metal.

13. A method of manufacturing a total disc replacement system, comprising the steps of:
(a) manufacturing a first anchor plate to have a first and second generally flat surface;
(b) manufacturing a second anchor plate to have a first and second generally flat surface; and
(c) manufacturing an internal assembly to include a first bearing surface, a second bearing surface, and an elastomeric polymer.

14. The method of claim 13, wherein step (a) includes manufacturing said first anchor plate to include an anchor element for placement into a first vertebral body, said anchor element extending in a generally perpendicular manner from said first generally flat surface, and step (b) includes manufacturing said second anchor plate to include an anchor element for placement into a second vertebral body, said second anchor element extending in a generally perpendicular manner from said second generally flat surface.

15. The method of claim 14, wherein each of said anchor elements includes a plurality of antimigration features.

16. The method of claim 15, wherein said plurality of antimigration features comprise a plurality of teeth.

17. The method of claim 13, wherein step (b) includes manufacturing said first anchor plate to include a post dimensioned to interact with said first bearing surface, said post extending in a generally perpendicular manner from said second generally flat surface, and step (b) includes manufacturing said second anchor plate to include a post dimensioned to interact with said second bearing surface, said post extending in a generally perpendicular manner from said second generally flat surface.

18. The method of claim 13, wherein step (c) includes manufacturing said first bearing surface to include an aperture dimensioned to interact with a post extending from said second generally flat surface of said first anchor plate.

19. The method of claim 13, wherein step (c) includes manufacturing said second bearing surface to include an aperture dimensioned to interact with a post extending from said second generally flat surface of said second anchor plate.

20. The method of claim 13, wherein said internal assembly is manufactured such that said elastomeric polymer comprises a hydrogel.

21. The method of claim 13, wherein said internal assembly is manufactured such that said elastomeric polymer comprises a cross-linked hydrogel.

22. The method of claim 13, wherein said internal assembly is manufactured such that said elastomeric polymer comprises at least one of a naturally-occurring polymer, a synthetic polymer, and a mixture of naturally-occurring and synthetic polymers.

23. A total disc replacement system, comprising:
a first anchor plate having a first and second generally flat surface, wherein said first anchor plate includes an anchor element for placement into a first vertebral body and a post element, said anchor element extending in a generally perpendicular manner from said first generally flat surface, and said post element extending in a generally perpendicular manner from said second generally flat surface;
a second anchor plate having a first and second generally flat surface, wherein said second anchor plate includes an anchor element for placement into a second vertebral body and a post element, said anchor element extending in a generally perpendicular manner from said first generally flat surface, and said post element extending in a generally perpendicular manner from said second generally flat surface; and
an internal assembly disposed between said first and second anchor plates, said internal assembly including a first bearing surface, a second bearing surface, and an elastomeric polymer situated between said first and second bearing surfaces, wherein said first and second bearing surfaces each include an aperture dimensioned to receive said post elements on said first and second anchor plates.
